(19) United States
(12) Patent Application Publication
(10) Pub. No.: US 2006/0247542 A1
(43) Pub. Date: Nov. 2, 2006

(54) Fatigue degree estimating method, fatigue degree estimating device, and database

(76) Inventors: Yasuyoshi Watanabe, Osaka (JP); Hirohiko Kuratsune, Osaka (JP); Koji Yamaguchi, Kyoto (JP); Tetsuya Sasabe, Osaka (JP)

Correspondence Address: SHERIDAN ROSS PC, 1560 BROADWAY, SUITE 1200, DENVER, CO, 80202, US

(21) Appl. No.: 10/555,114

(22) PCT Filed: Jun. 25, 2004
(86) PCT No.: PCT/JP04/09034

§ 371 Date: May 24, 2006

(30) Foreign Application Priority Data
Jun. 27, 2003 (JP) 2003-185156

(51) Int. Cl. A61B 5/02

(52) U.S. Cl. 600/500; 600/483

(57) ABSTRACT

The present invention provides a method for evaluating the degree of fatigue of a human body. The evaluation method of the present invention is characterized by evaluating the degree of fatigue of a human body by using, as an index, change in waveform of a pulse wave, particularly acceleration pulse wave.

### TECHNICAL FIELD

The present invention relates to a method for evaluating the degree of fatigue of a human body, a fatigue evaluation apparatus, and database. More specifically, the present invention relates to a fatigue evaluation method using, as an index, change in waveform of a pulse wave, especially acceleration pulse wave, fatigue evaluation apparatus, and database.

### BACKGROUND ART

For many people living in the modern world, fatigue is a phenomenon they feel in daily life. For example, according to the result of the survey on fatigue conducted in 1999 by department of epidemiology, National Institute of Public Health in Japan, it is obvious that 59.1% of the respondents said “they felt fatigue now”.

Originally, Japanese people complain of fatigue and shoulder stiffness at a much higher percentage than Western people. For example, as for sales of drugs and quasi-drugs featuring nutritional fortification and muscle fatigue treatment, Japan is the best in the world far ahead of all other countries. In addition, costs of compresses for shoulder stiffness of Chinese herbal medicines featuring fatigue-treating effects are covered by insurance. In view of this, it can be said that Japan is a “world' leading fatigue country”.

Of course, there are many people who complain of fatigue in other countries, apart from Japan. Therefore, it can be said that fatigue alleviation and overcoming is one of the most important problems in the modern world. However, research on fatigue is under development.

For example, as described above, although fatigue has been pervasive in Japan, almost no research on fatigue was conducted in Japan until a few years ago.

As for fatigues, the disease termed chronic fatigue syndrome (CFS) has been seen as a problem (For example, see non-patent document 1). The CFS commonly develops suddenly in a healthy person when he or she suffers from a disease such as cold. Examples of the symptoms include unexplained severe general malaise, slight fever, headache, lymphadenopathy, muscular pain, feeling of lassitude, thinking and concentration impairment, depressive symptom, and, sleep disorder. These symptoms last for years. However, the cause of the CFS has not been determined scientifically.

Further, examples of the problem involving fatigue include an overwork death that is widely known and is coming to the fore as a social issue in present-day Japan. The overwork death refers to a sudden death from overworking. Although the overwork death is recognized as a serious issue from medical, economic, and social standpoints, a scientific mechanism for the overwork death has been almost unclear.

In order to proceed the research on fatigue, including prevention against the CFS and overwork death and treatment for the CFS and overwork death, it is necessary to medically grasp, i.e. reproducibly quantify “state of fatigue”, which is a underlying cause of the CFS and overwork death.

Generally, for determination of pathologic state, various physiological phenomena involved in human's vital activity are widely used as indices by quantifying them in some way. Therefore, it is considered possible to quantify “the degree of fatigue” by finding out some kind of index for fatigue.

By the way, as one of indices for the evaluation of a cardiocirculatory system, a pulse wave is known. The pulse wave is pulsation of peripheral arteries and veins which occurs with heart contractions and expansions, and includes much information about hemodynamics from central blood vessels to peripheral blood vessels. That is, when a flow of blood pumped from the heart is transmitted as a pulse to peripheral vessels, physiological conditions such as heart rate, hemodynamics, and condition changes in arterioles create distortions in pulse waveforms. Various techniques have been proposed for individually evaluating such distortions contained in the pulse wave for the evaluation of a cardiocirculatory system.

Specifically, for example, a non-patent document 2 discloses a technique of a test using finger plethysmograph (DPG). DPG indicates a difference between the amount of arterial blood input and the amount of venous blood input in a local part, which allows for estimation with great accuracy of pressure pulse wave of an artery around the local part. Examples of a target to be evaluated by DPG include peripheral blood circulation and autonomic nervous system function.

However, the DPG has the problems of unstable baseline, fewer undulations of its waveform, and difficulty in evaluation of inflection points. In view of this, a technique has been proposed for differentiating a waveform, aiming to support the analysis of the DPG.

For example, non-patent document 3 discloses a research for analysing a feature of DPG waveform of various diseases, such as lung function disorder, hypertension, and arteriosclerotic heart disease, together with a first derivative waveform of the DPG and ballistocardiogram (heart function test) recorded at the same time. This research indicates that the DPG waveform or the first derivative waveform of the DPG responds strongly to a peripheral artery system.

Further, in recent years, acceleration plethysmogram has been proposed. The acceleration plethysmogram is a second derivative waveform obtained by differentiating the first derivative of the DPG waveform. As an example of a technique relating to the acceleration plethysmogram, techniques disclosed in non-patent document 4 and patent document 1 can be given.

In addition, it is known that a pulse wave is applicable to the evaluation of systems other than cardiocirculatory system. Specifically, for example, patent document 2 discloses a evaluation method using a waveform of acceleration pulse wave, and a result of research on a factor uniquely defining the waveform and a modifying factor. Also, the patent document 2 describes that acceleration pulse wave is applicable to aging of blood vessels and the evaluation of physical condition.

Patent document 3 discloses a stress level measuring method using a pulse wave. As to this technique, an apparatus for outputting a stress level by using a common expression “parameter” is disclosed. Further, patent documents 4 and 5 disclose a physical condition determining method and a physical condition determining apparatus both of which determines a physical condition as a result of comparison between a pulse wave relative to oxygenated hemoglobin and a pulse wave relative to non-oxygenated hemoglobin.

Further, non-patent documents 5 and 6 discloses findings on state-dependence of a chaos attractor of finger plethysmogram. Specifically, it is known that a complexity of chaos decreases due to fatigue, stress, health deterioration, or aging, thereby causing a regularly and simply structured attractor.

[Patent Document 1]

Japanese Laid-Open Patent Application No. 217797/2000 (Tokukai 2000-217797; published on Aug. 8, 2000)

[Patent Document 2]

Japanese Laid-Open Patent Application No. 238867/2002 (Tokukai 2002-238867; published on Aug. 27, 2002)

[Patent Document 3]

Japanese Laid-Open Patent Application No. 51234/1995 (Tokukaihei 7-51234; published on Feb. 28, 1995)

[Patent Document 4]

Japanese Laid-Open Patent Application No. 272708/2002 (Tokukai 2002-272708; published on Sep. 24, 2002)

[Patent Document 5]

Japanese Laid-Open Patent Application No. 61921/2003 (Tokukai 2003-61921; published on Mar. 4, 2003)

[Non-Patent Document 1]

Journal of Japanese Society of Internal Medicine, Vol. 81, 573-582 (1992)

[Non-Patent Document 2]

Toshiko TAKEMIYA: clinical pulse wave, Journal of Tokyo Women's Medical University, Vol. 46, 1-12 (1976)

[Non-Patent Document 3]

Yutaka NISHIO: Derivative waveform of finger plethysmogram, pulse wave, 3(2), 127-130 (1973)

[Non-Patent Document 4]

Yuji SANO and others: Evaluation of circulation in blood vessels by using acceleration plethysmogram and its application, Science of Labor, 61(3), 129-143 (1985)

[Non-Patent Document 5]

Journal of Society of Biomechanisms Japan, Vol. 19, No. 2 (1995)

[Non-Patent Document 6]

Japanese Journal of Nursing Research, Vol. 34, No. 4, August in 2001

As described above, techniques for evaluating systems other than cardiocirculatory system by using a pulse wave or acceleration pulse wave are conventionally known. Also, techniques for evaluating a physical condition using a pulse wave or an acceleration pulse wave are known. However, a relation between the pulse wave or the acceleration pulse wave and fatigue is unknown.

For example, the patent document 3 discloses a technique for measuring stress levels, as described above, but is totally silent about fatigue. Besides, the patent document 3 has no specific descriptions about parameter calculation method, correspondence between the stress levels and clinical data, and others. Similarly, the patent documents 3 through 5 and the non-patent documents 5 and 6 are totally silent about a relation between a pulse wave and fatigue.

Further, regardless of a estimation target, it can be considered that more specific evaluation is realized by using an acceleration pulse wave rather than a pulse wave. However, the patent documents 3 through 5 and the non-patent documents 5 and 6 are totally silent about acceleration pulse wave.

Here, the patent document 2 discloses a technique using a parameter of a wave component in acceleration pulse wave. However, this technique evaluates aging of blood vessels by using blood vessel aging score derived from an average value and a standard deviation of two waveform indices, and thus needs an extremely complex analysis.

In addition, the non-patent documents 5 and 6 disclose findings on a chaos attractor of finger plethysmogram and dependence on a state such as health condition. However, they have no disclosure and suggestions about association between a chaos attractor of acceleration pulse wave and fatigue.

### DISCLOSURE OF INVENTION

As a result of extensive research in view of the above problem, inventors of the present invention found out that it is possible to evaluate fatigue easily and quantitatively by using a pulse wave, particularly acceleration pulse wave, without requirement for a special analysis, and completed the present invention.

Specifically, the inventors found out that it is possible to measure the degree of fatigue easily and quantitatively by measuring a wave height of at least one of wave components a, b, c, d, and e in a waveform of acceleration plethysmogram that is a second derivative of a finger plethysmogram (DPG) in a pulse wave, particularly by measuring a wave height of the wave component a, and completed the present invention.

Further, the inventors found out that it is possible to measure the degree of fatigue easily and quantitatively by performing chaos analysis on the acceleration pulse wave, and completed the present invention.

That is, the present invention relates to:

(1) a method for evaluating the degree of fatigue by using as an index change in waveform of a pulse wave; preferably the method of using acceleration pulse wave as a pulse wave; specifically, a fatigue evaluation method using, as an index, change in waveform of at least one of wave components, a, b, c, d, and e in acceleration pulse wave, wherein it is evaluated that a wave height lower than a wave height at a reference time is indicative of fatigue, and wherein change in waveform of the acceleration pulse wave as an index is change of a ratio in measured value between at least two of the wave components a, b, c, d, and e in the acceleration pulse wave, and the measured value of the wave component is at least one of measured values: wave height, frequency, wavelength, cycle, and variation coefficient of the foregoing measured values;

(2) a method for collecting data which is an object to be evaluated for the degree of fatigue, wherein change of a measured value of at least one of wave components a, b, c, d, and e in acceleration pulse wave is measured, and the measured value is at least one of measured values: wave height, frequency, wavelength, cycle, and variation coefficient of the foregoing measured values; preferably a method for collecting data which is an object to be evaluated for the degree of fatigue, wherein change in a wave height of at least one of wave components a, b, c, d, and e in acceleration pulse wave is measured;

(3) a database which includes a measured value of at least one of wave components a, b, c, d, and e in acceleration pulse wave at a reference time, and the measured value is at least one of measured values: wave height, frequency, wavelength, cycle, and variation coefficient of the foregoing measured values; preferably, the database which includes a wave height of at least one of the wave components a, b, c, d, and e in acceleration pulse wave at a reference time; and

(4) a fatigue evaluation method for evaluating a degree of fatigue by using, as an index, change in waveform of at least one of wave components a, b, c, d, and e in acceleration pulse wave, wherein it is evaluated that a wave height lower than wave height data stored in the foregoing database is indicative of fatigue.

Further, the present invention includes the following method or apparatus:

(5) a method wherein chaos analysis is performed on the acceleration pulse wave so that a degree of fatigue is evaluated by using, as an index, change of a factor in the chaos analysis; preferably, the method wherein the factor in the chaos analysis used as the index is a maximum Lyapunov exponent, and it is evaluated that the maximum Lyapunov exponent lower than a maximum Lyapunov exponent at a reference time is indicative of fatigue. Further, preferably, the method wherein the factor in the chaos analysis used as the index is a correlation dimension, and it is evaluated that the correlation dimension closer to an integral value than a correlation dimension at a reference time, is indicative of fatigue; preferably, the method wherein a maximum entropy method is used in the chaos analysis; preferably, the method wherein the factor in the chaos analysis used as the index is a high-frequency component, and it is evaluated that the high-frequency component having a sharper slope than a high-frequency component at a reference time is indicative of fatigue.

Note that, the above fatigue evaluation method may be performed by using a pulse wave obtained from a subject.

Note that, the pulse wave obtained from a subject is obtained by measurement from artery of fingertip, earlobe, wrist, upper arm, or carotid, for example. However, a pulse wave may be measured from any body part where a pulse wave can be measured. The body part from which a pulse wave is measured is not particularly limited.

Further, (6) a fatigue evaluation apparatus comprising: evaluation means for evaluating a degree of fatigue by using, as an index, change in waveform of a pulse wave obtained from a subject. A fatigue evaluation apparatus comprising: evaluation means for evaluating a degree of fatigue by using, as an index, change in waveform of acceleration pulse wave determined on the basis of a pulse wave obtained from a subject; preferably, the apparatus further comprising acceleration pulse wave determining means for determining acceleration pulse wave by twice differentiating the pulse wave obtained from the subject. Further, preferably, the apparatus wherein the evaluation means evaluates the degree of fatigue by using, as an index, change in waveform of at least one of wave components a, b, c, d, and e. 19. Still further, preferably, the apparatus wherein the evaluation means evaluates the degree of fatigue by using, as the index, change of a measured value of at least one of wave components a, b, c, d, and e in the acceleration pulse wave, and the measured value of the wave component is at least one of measured values: wave height, frequency, wavelength, cycle, and variation coefficient of the foregoing measured values. Yet further, preferably, the apparatus wherein the evaluation means evaluates the degree of fatigue by using, as an index, change in a wave height of the wave component a in the acceleration pulse wave.

Further, the apparatus is preferably such that the evaluation means evaluates the degree of fatigue by using, as an index, change in waveform of at least one of wave components, a, b, c, d, and e in acceleration pulse wave, wherein it is evaluated that a wave height lower than a wave height at a reference time is indicative of fatigue. Still further, it is preferable that the evaluation means evaluates the degree of fatigue by using, as an index, change in a wave height of the wave component a, and it is evaluated that a wave height lower than a wave height at a reference time is indicative of fatigue.

Further, the apparatus is preferably such that the evaluation means evaluates the degree of fatigue by using, as the index, change of a ratio in measured value between at least two of wave components a, b, c, d, and e in the acceleration pulse wave, and the measured value of the wave component is at least one of measured values: wave height, frequency, wavelength, cycle, and variation coefficient of the foregoing measured values.

Further, (7) a fatigue evaluation apparatus comprising: chaos analyzing means for performing chaos analysis on an acceleration pulse wave determined on the basis of a pulse wave obtained from a subject; and evaluation means for evaluating the degree of fatigue by using, as an index, change of a factor in the chaos analysis; preferably, the apparatus wherein the factor in the chaos analysis used as the index is a maximum Lyapunov exponent, and the evaluation means evaluates that the maximum Lyapunov exponent lower than a maximum Lyapunov exponent at a reference time is indicative of fatigue. Further, preferably, the apparatus wherein the factor in the chaos analysis used as the index is a correlation dimension, and the evaluation means evaluates that the correlation dimension closer to an integral value than a correlation dimension at a reference time, is indicative of fatigue. Still further, preferably, the apparatus wherein the analyzing means uses a maximum entropy method in the chaos analysis. Yet further, the apparatus wherein the factor in the chaos analysis used as the index is a high-frequency component, and the evaluation means evaluates that the high-frequency component having a sharper slope than a high-frequency component at a reference time is indicative of fatigue.

For a fuller understanding of the nature and advantages of the invention, reference should be made to the ensuing detailed description taken in conjunction with the accompanying drawings.

### BRIEF DESCRIPTION OF DRAWINGS

FIG. 1 is a diagram illustrating acceleration plethysmogram's typical five wave components a, b, c, d, and e. A longitudinal axis represents a wave height (amplitude (mV)), and a lateral axis represents a time (sec).

FIG. 2 is a graph illustrating change in wave height of the wave component a in acceleration pulse wave before and after fatigue loading.

FIG. 3 is an explanatory view of frequency spectrum analysis.

FIG. 4 is an explanatory view illustrating a slope of a high-frequency component obtained by frequency spectrum analysis.

FIG. 5 is a view illustrating a fatigue test schedule in Examples.

FIG. 6 is a view illustrating a VAS test sheet.

FIG. 7 is a view illustrating a Face Scale test sheet.

FIG. 8 is a view illustrating ways of mental fatigue loading.

FIG. 9 is a view illustrating ways of physical fatigue loading.

FIG. 10 is a view illustrating a result of VAS.

FIG. 11 is a view illustrating a result of Face Scale.

FIG. 12 is a view illustrating a result of determining a maximum Lyapunov exponent before and after fatigue loading.

FIG. 13 is a view illustrating a result of determining a correlation dimension before and after fatigue loading.

FIG. 14 is a view illustrating a result of determining a slope of a high-frequency component before and after fatigue loading.

FIG. 15 is a view illustrating a functional block of a fatigue evaluation system according to one embodiment of the present invention.

FIG. 16 is a view illustrating an exemplary process flow of a fatigue evaluation apparatus according to one embodiment of the present invention.

FIG. 17 is a view illustrating a functional block of a fatigue evaluation system according to another embodiment of the present invention.

FIG. 18 is a view illustrating another exemplary process flow of a fatigue evaluation apparatus according to one embodiment of the present invention.

FIG. 19 is a view illustrating another exemplary process flow of a fatigue evaluation apparatus according to one embodiment of the present invention.

### BEST MODE FOR CARRYING OUT THE INVENTION

(1) Method and Apparatus for Evaluating the Degree of Fatigue by Using Change in Waveform of a Pulse Wave, as an Index

In the present invention, the degree of fatigue refers to “the degree of temporal degradation in physical and mental performances under continuous physical and mental loads”. The “degradation in performances” refers to “the degree of qualitative or quantitative degradation in physical and mental working abilities.

In recent years, a disease termed chronic fatigue syndrome (hereinafter referred to as CFS) has been seen as a problem (Journal of Japanese Society of Internal Medicine, Vol. 81, 573-582 (1992)). The CFS commonly develops suddenly in a healthy person when he or she suffers from a disease such as cold. Examples of the symptoms include unexplained severe general malaise, slight fever, headache, lymphadenopathy, muscular pain, feeling of lassitude, thinking and concentration impairment, depressive symptom, and, sleep disorder. These symptoms last for years. In addition, there is the problem of an overwork death that is widely known and is coming to the fore as a social issue in present-day Japan. The overwork death refers to a sudden death from overworking. Although the overwork death is recognized as a serious issue from medical, economic, and social standpoints, a scientific mechanism for the overwork death has been almost unclear.

A method according to the present invention realizes to easily and quantitatively measure the degree of fatigue. Therefore, it can be considered that quantification of the degree of fatigue is important in terms of prevention against the CFS and overwork death and treatment for the CFS and overwork death.

A waveform of a pulse pressure (pulse wave), which is difference between systolic blood pressure and diastolic blood pressure, changes due to resonance caused by synthesis of a projected wave and a reflected wave in various sections in the course of traveling from a main artery to peripheral arteries. The degree of the change can be regarded as the sum total of influence on conditions and properties of blood vessels. Many plethysmographs in current use are photoelectric finger plethysmographs. The principle of the plethysmograph is based on a method of obtaining a waveform in such a manner that a fingertip is irradiated with light of a wavelength having a specific property of light absorption to hemoglobin to find the change in volume of blood flow in blood vessels from an absorbed light or a reflected light. There has been no report on a correlation between such a pulse wave and human's fatigue.

A measured value of a pulse wave as an index is a waveform, frequency, wavelength, wave height, cycle, or variation coefficient of the foregoing measured values.

In a preferable mode, the present invention uses an acceleration pulse wave as a pulse wave that is the index.

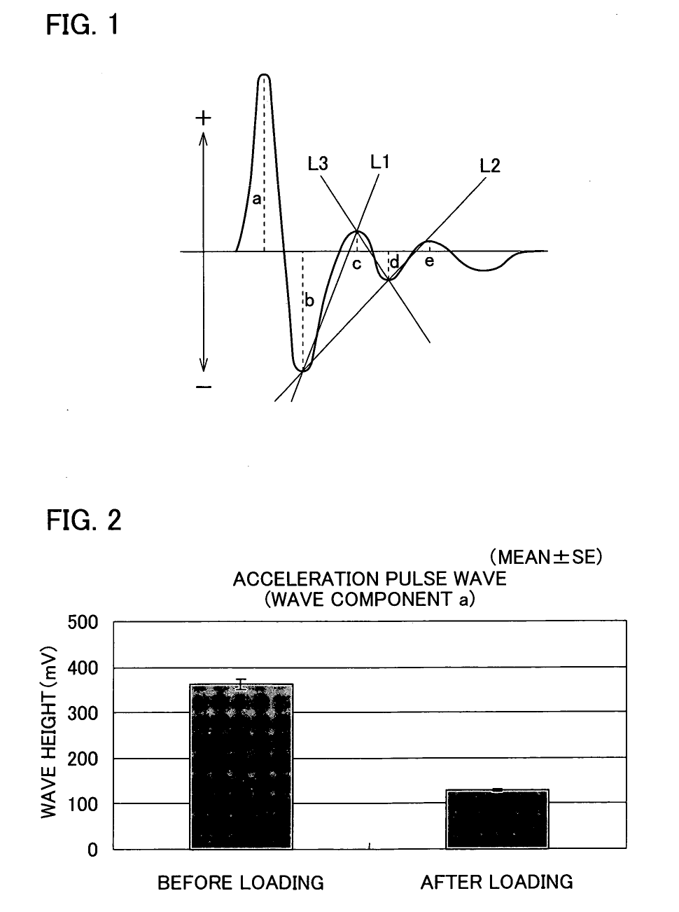

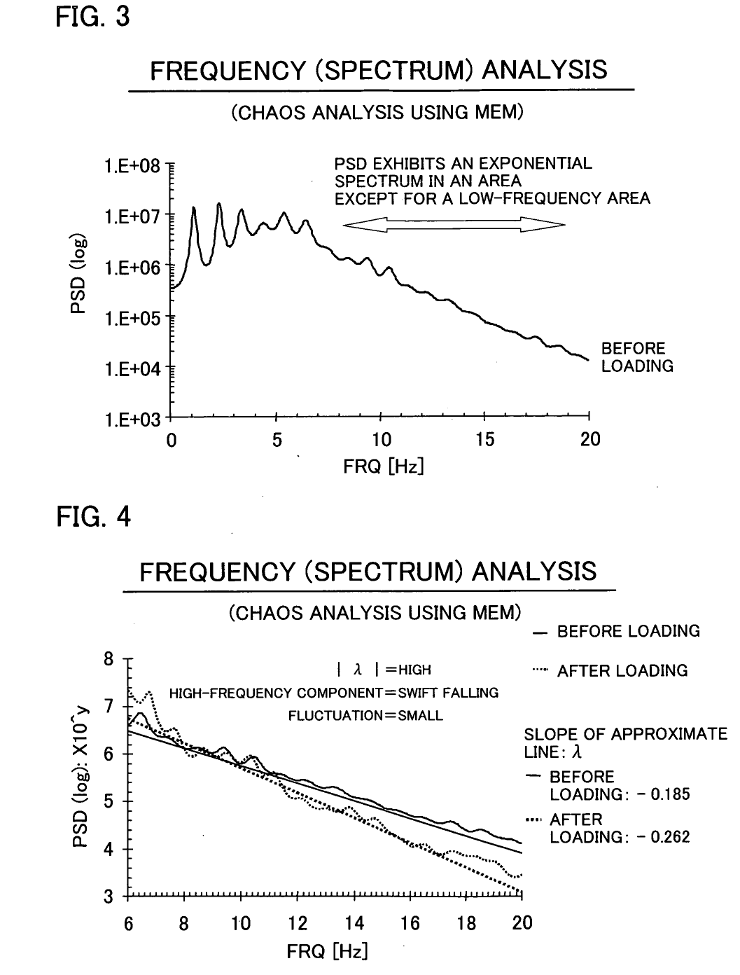

The “acceleration plethysmogram” is a second derivative of finger plethysmogram (DPG) produced by a plethysmograph. It is considered that the acceleration plethysmogram emphasizes an inflection point of its waveform for ease of evaluation of the waveform so as to grasp blood circulatory movement. As an inflection point of an original waveform is sharper, an inflection point of a second derivative of the original waveform is higher (Document: Ayu SUZUKI (1991): physiological function test, pulse wave, acceleration pulse wave, Gendai Iryo, 23(1), 61-65.). This realizes easy pattern reading and measurement of the waveform with the inflection point. That is why the acceleration plethysmogram is more suitable for a research on relation with physiological functions and hemodynamics. An acceleration pulse waveform is a waveform in systole and consists of five wave components a, b, c, d, and e (FIG. 1). In FIG. 1, a, longitudinal axis represents a wave height (amplitude (mV)).

In more preferable mode, a waveform of acceleration pulse wave used as an index is a waveform of one of the wave components a, b, c, d, and e, more preferably a waveform of the wave component a. Changes in waveform of a pulse wave used as an index are changes in waveform, frequency, wavelength, wave height, cycle, and variation coefficient of the foregoing measured values. The present invention includes a method in which change in waveform of acceleration plethysmogram used as an index is change of a ratio in measured value between at least two of the wave components a, b, c, d, and e in acceleration plethysmogram, and the measured value of the wave component is at least one of the following measured values: wave height, frequency, wavelength, cycle, and variation coefficient of the foregoing measured values.

The acceleration plethysmogram is a second derivative of DPG produced by a plethysmograph. For the acceleration plethysmogram, a peripheral pulse wave is measured because a peripheral pulse waveform has more raised and recessed portions than a central pulse waveform, which realizes easy reading of the waveform.

The present invention provides, as a typical mode, a fatigue evaluation method using, as an index, change in waveform of at least one of the wave components a, b, c, d, and e in the acceleration pulse wave, wherein it is evaluated that a wave height lower than a wave height at a reference time is indicative of fatigue. The reference time is a time when a subject has no fatigue or a time before he or she is put under fatigue loads.

Further, the present invention provides a method in which chaos analysis is performed on acceleration pulse wave, determined on the basis of a pulse wave obtained from a subject, and a factor in the chaos analysis chaotic parameter) of this chaos analysis is used as an index for evaluation of the degree of fatigue which the subject suffers from.

Chaos, a deterministic mechanism, is a system which exhibits extremely complex changes due to its nonlinearilty and a system whose behavior is extremely difficult to predict although it is a determinism. At present, it is said that almost all phenomena of nature can be represented by chaos. Chaos analysis is a method for analyzing the chaos. Now, there are the following two types of chaos analyses:

Traditional chaos analysis using a combination of correlation dimension analysis and maximum Lyapunov exponent analysis; and

Chaos analysis using maximum entropy method.

The following will describe the above two analyses and their factors.

First, “traditional chaos analysis using a combination of correlation dimension analysis and maximum Lyapunov exponent analysis” is described below.

The “traditional chaos analysis using a combination of correlation dimension analysis and maximum Lyapunov exponent analysis” has been actively adopted for the recent tens of years, and primarily uses a combination of correlation dimension analysis and maximum Lyapunov exponent analysis.

In this analysis, necessary conditions for chaos are:

(i) correlation dimension is non-integral number; and

(ii) maximum Lyapunov exponent is positive.

The above two conditions must be fulfilled concurrently.

Now, the “correlation dimension” is one of measures of Fractal Dimension and is calculated from a correlation integral equation represented by the following equation (1):

$$C ( r ) = \frac{1}{{n}^{2}} {{\sum }_{i , j = 1}^{n}}_{i \neq j} H ( r - \vert {x}_{i} - {x}_{j} \vert ) . \qquad ( 1 )$$

As the correlation dimension is higher, a graphical plot of time-series data becomes more complex. That is, this is a measure of whether an event happens repeatedly (measure of self-similarity). Specifically, the correlation dimension of integral number indicates a high possibility that the same event happens repeatedly. On the other hand, the correlation dimension of non-integral number indicates that the same event does not happen repeatedly.

The “maximum Lyapunov exponent” is a measure of initial-value dependency or long-term prediction impossibility, and expressed by the following equation (2):

$$λ = {lim}_{T -> ∞} \frac{1}{T} {\sum }_{t}^{T - 1} log \vert \frac{ⅆ y ( t + 1 )}{ⅆ y ( t )} \vert . \qquad ( 2 )$$

Increase of the maximum Lyapunov exponent indicates that an event exhibits totally different behaviors although difference from an initial value is very small.

In the situation where the correlation dimension is non-integral number, as the maximum Lyapunov exponent is higher, long-term prediction becomes more difficult. The traditional chaos analysis assumes that the higher the maximum Lyapunov exponent is, an event is more chaotic. Note that, in the present invention, as is stated in Examples described later, it can be evaluated that the correlation dimension closer to an integral value is indicative of more fatigue, and it can be evaluated that the lower maximum Lyapunov exponent is indicative of more fatigue.

Next, the “chaos analysis using maximum entropy method” will be described below.

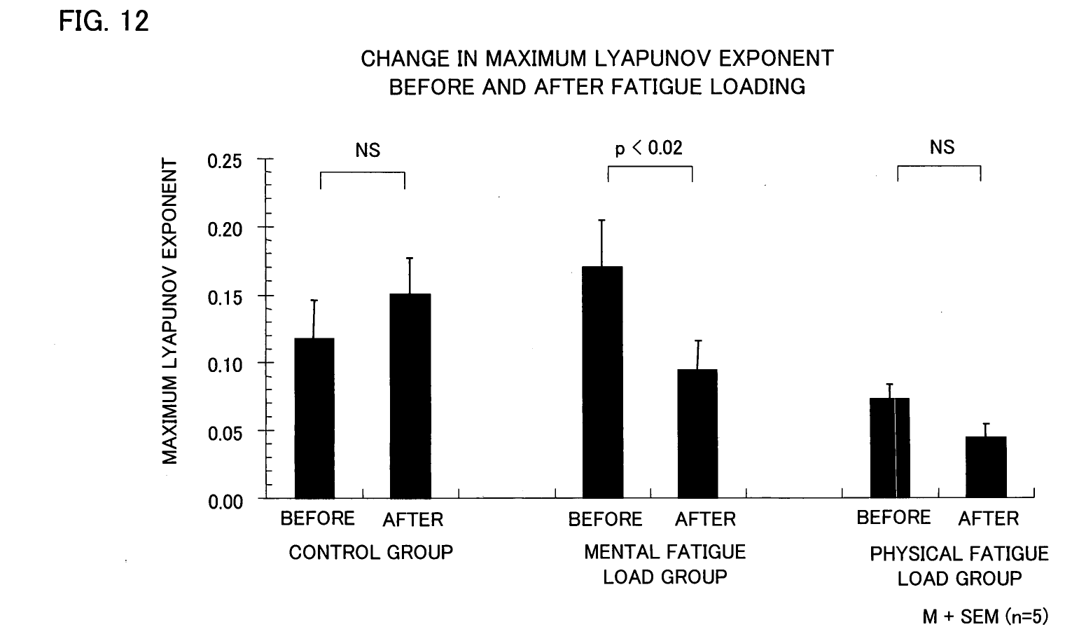

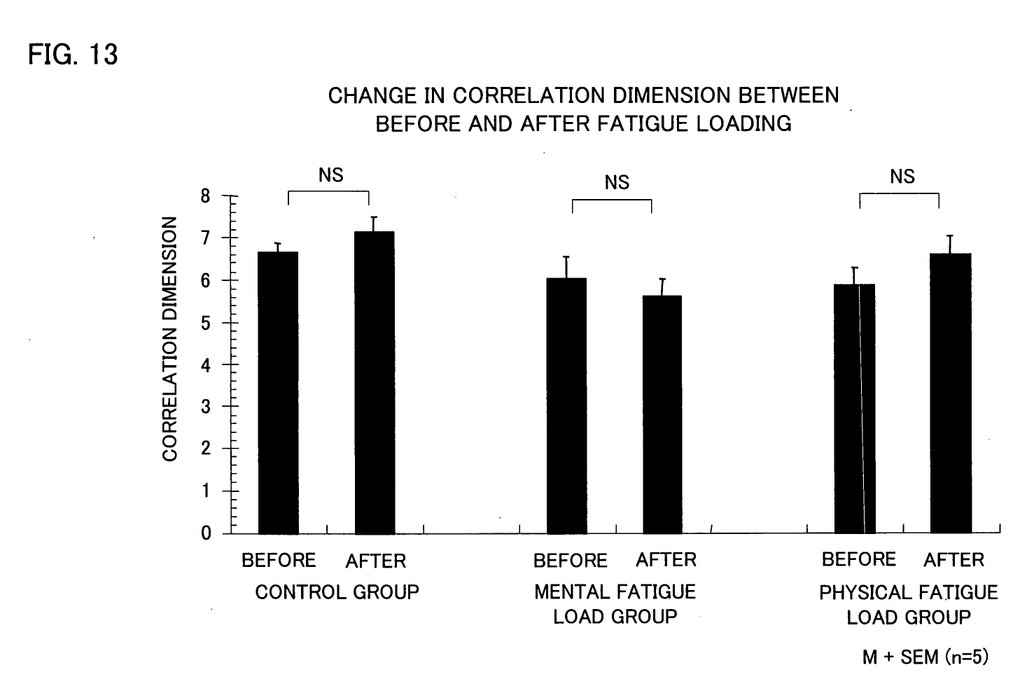

The “chaos analysis using maximum entropy method” is a method for analyzing chaotic nature of an event using the maximum entropy method that is one of frequency analyses, and is a method whose theoretical background has been established for the recent years. In fast Fourier transformation which is used commonly in the field of frequency analysis, the chaos analysis using the maximum entropy method allows for chaos analysis of an event which happens in a relatively short time, which was used to be difficult. The chaos analysis using the maximum entropy method utilizes an exponentially falling high-frequency component, except for low-frequency component, that is one of four prerequisites to time-series data being chaos. A sharper slope of the high-frequency component indicates reduced chaotic nature, that is, disappearance of fluctuation component (See FIGS. 12 and 13).

Note that, in the present invention, as is stated in Examples described later, it can be evaluated that the high-frequency component having a sharper slope is indicative of fatigue.

In the fatigue evaluation method according to the present invention, the four prerequisites for chaos is as follows:

(i) PSD exhibits an exponential spectrum in an area except for a low-frequency area;

(ii) fundamental mode and higher harmonics are observed;

(iii) subharmonics are observed in the process of chaos' growth; and

(iv) inverse cascade are observed in the process of chaos' development.

Reference: N. Ohtomo, T. Kamo, M. Watanabe, K. Yoneyama, Y. Tanaka and R. Hayashi, “Power Spectral Densities of Temporal Variations of Blood Pressures”, Japanese Journal of Applied Physics, Vol. 35 (1996) pp. 5571-5582

In a method according to the present invention, (i) is adopted among these prerequisites. It can be considered that acceleration pulse wave time-series data has chaotic nature because the high-frequency component falls exponentially, except for a low-frequency component. Because of the high-frequency component falling exponentially, the acceleration pulse wave time-series data becomes a straight line in its semilogarithmic graph having an x-axis representing frequency and a y-axis representing PSD. In view of this, the method of the present invention applies a slope of the straight line to quantification of chaotic nature of acceleration pulse wave. It is indicated that as the slope is sharper (absolute value of the slope is higher), chaotic nature reduces more, i.e. fluctuation component disappears (See FIGS. 12 and 13).

Note that a fatigue evaluation method according to the present invention can be also performed by using a pulse wave obtained from a subject. That is, in the present invention, the degree of fatigue of a subject can be evaluated by using a pulse wave obtained by a process of obtaining a pulse wave with the touch on the body of the subject. The process of obtaining a pulse wave is performed beyond the scope of the present invention.

In the above mode, the present invention also provides an apparatus for implementing an evaluation method of the present invention. The apparatus includes: a section (means) for measuring human's pulse wave; a member (means) for determining acceleration pulse wave from the pulse wave if necessary; a section (means) for performing a predetermined analysis on the acceleration pulse wave for evaluation; a section (means) for generating an image based on the measured data; a section (means) for displaying an image; and other sections.

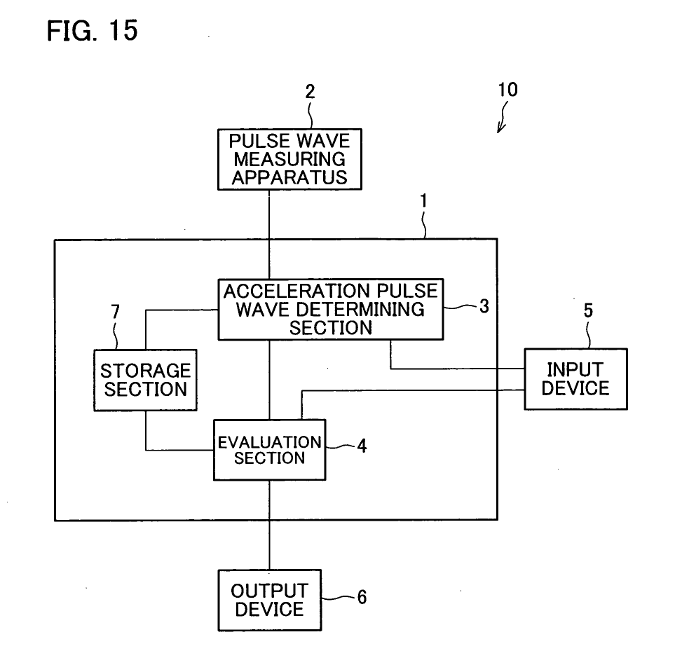

For example, as illustrated in FIG. 15, a fatigue evaluation system 10 according to the present embodiment, includes: a pulse wave measuring apparatus 2 for measuring a pulse wave of a subject; a fatigue evaluation apparatus 1; an input device 5; and an output device 6. The fatigue evaluation apparatus 1 includes an acceleration pulse wave determining section 3, an evaluation section 4, and a storage section 7.

For the pulse wave measuring apparatus 2, used is an apparatus for measuring (determining) a conventionally known pulse wave, but the pulse wave measuring apparatus 2 is not particularly limited to this. For example, an apparatus for determining a conventionally known finger plethysmogram (DPG) (e.g. plethysmograph) can be adopted favorably.

The acceleration pulse wave determining section 3 determines acceleration pulse wave that is a second derivative of a pulse wave obtained by the pulse wave measuring apparatus 2. A specific structure of the acceleration pulse wave determining section 3 is not particularly limited. A conventionally known computing unit can be used.

The evaluation section 4 evaluates the degree of fatigue by using, as an index, change in waveform of the acceleration pulse wave determined by the acceleration pulse wave determining section 3. In other words, the evaluation section 4 is a section for implementing the foregoing fatigue evaluation method according to the preset invention.

The input device 5 is particularly not limited, provided that it is capable of inputting information on operations of the fatigue evaluation apparatus 1. As the input device 5, conventionally known input means such as keyboard, tablet, or scanner can be adopted favorably.

The output device 6 is display means for displaying various kinds of information such as information and results regarding operations of the fatigue evaluation system 10, including pulse wave, acceleration pulse wave, and results of evaluation. Specifically, as the output device 6, various kinds of display devices such as known CRT display, or liquid crystal display are used favorably. However, this is not the only possibility.

The output device 6 may record (printing/imaging) on a recording material, such as PPC sheet, various kinds of information that can be displayed on the display means. Specifically, an image forming apparatus, such as known ink jet printer or laser printer, is used favorably. However, this is not the only possibility.

That is, the output device 6 is means for outputting various kinds of information in the form of soft copy, and/or means for outputting various kinds of information in the form of hard copy. Note that, output means used in the present invention is not limited to the above display means and printing means. Alternatively, other output means may be included.

The storage section 7 stores various kinds of information (pulse wave, acceleration pulse wave, control information, results of evaluation, and other information) used in the fatigue evaluation system 10. Specifically, as the storage section 7, preferably used are conventionally known various storage means including: a semiconductor memory such as RAM or ROM; a magnetic disk such as flexible disk or hard disk, a disk such as optical disk including CD-ROM/MO/MD/DVD; and a card such as IC card (including a memory card) and optical card.

The storage section 14 may be integrated with the fatigue evaluation system 10 into one unit. Alternatively, the storage section 14 may be an external storage device that is provided separately from the fatigue evaluation system 10. Further, both the integrated storage section 7 and an external storage device may be provided. Examples of the integrated storage section 7 include an internal-type hard disk, flexible disk drive incorporated into a unit, CD-ROM drive, and DVD-ROM drive. Examples of the external storage device include an external-type hard disk and the foregoing disk drives of external-type.

Next, the following will describe specific functions of the evaluation section 4 which is a feature of the present invention. For example, the evaluation section 4 evaluates the degree of fatigue by using, as an index, change in waveform of at least one of wave components a, b, c, d, and e in acceleration pulse wave. Further, the evaluation section 4 evaluates the degree of fatigue by using, as an index, change of a measured value of at least one of wave components a, b, c, d, and e in acceleration pulse wave, and the measured value may be at least one of measured values: wave height, frequency, wavelength, cycle, and variation coefficient of the foregoing measured values. Particularly, the degree of fatigue is preferably evaluated by using, as an index, change in wave height of the wave component a in acceleration pulse wave.

Further, the evaluation section 4 evaluates the degree of fatigue by using, as an index, change in waveform of at least one of wave components a, b, c, d, and e in acceleration pulse wave, and evaluates that a wave height lower than a wave height at a reference time is indicative of fatigue. Particularly, the evaluation section 4 preferably evaluates the degree of fatigue by using, as an index, change in wave height of the wave component a, and evaluates that the wave height of the wave component a lower than a wave height at a reference time is indicative of fatigue.

Further, the evaluation section 4 evaluates the degree of fatigue by using, as an index, change of a ratio in measured value between at least two of wave components a, b, c, d, and e in acceleration pulse wave, and the measured value may be at least one of measured values: wave height, frequency, wavelength, cycle, and variation coefficient of the foregoing measured values.

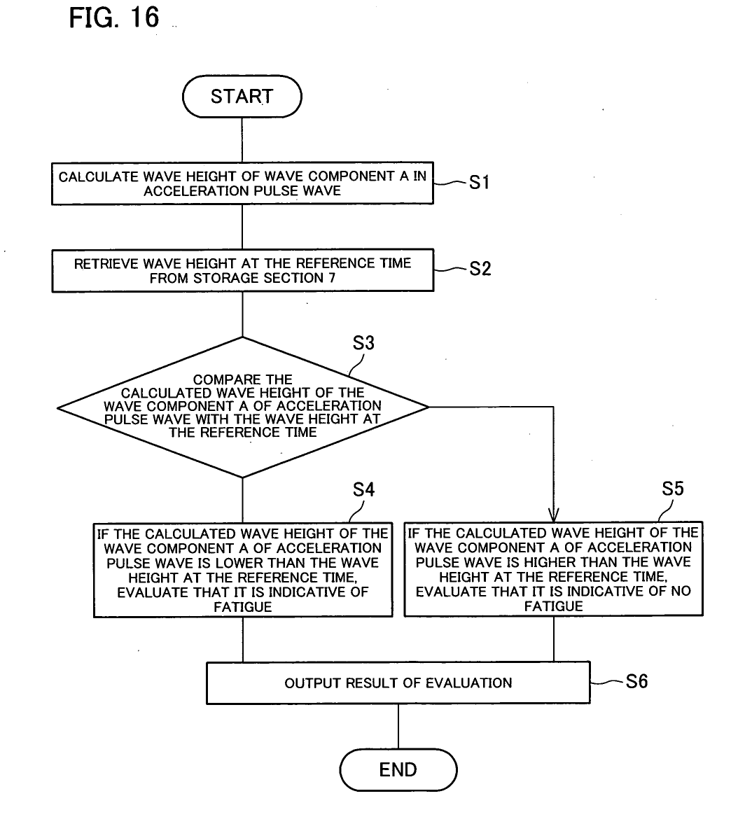

The following will describe one example of a specific process flow in the evaluation section 4. FIG. 16 illustrates an exemplary process flow of a case when the evaluation section 4 evaluates the degree of fatigue by using, as an index, change in wave height of the wave component a in acceleration pulse wave, it evaluates that the wave height of the wave component a lower than a wave height at a reference time is indicative of fatigue.

In Step 1, the evaluation section 4 calculates a wave height of the wave component a in acceleration pulse wave. In Step 2, the evaluation section 4 retrieves a wave height at the reference time from the storage section 7. Then, the evaluation section 4 compares the calculated wave height of the wave component a of acceleration pulse wave with the wave height at the reference time (Step 3). The evaluation section 4 evaluates, if the calculated wave height of the wave component a of acceleration pulse wave is lower than the wave height at the reference time, that it is indicative of fatigue (high degree of fatigue) (Step 4). On the other hand, the evaluation section 4 evaluates, if the calculated wave height of the wave component a of acceleration pulse wave is higher than the wave height at the reference time, that it is indicative of no fatigue (low degree of fatigue) (Step 5). Finally, the evaluation section 4 outputs a result of the evaluation to the output device 6.

The above descriptions are given herein based on the case where the degree of fatigue is evaluated by using, as an index, the wave height of the wave component a of acceleration pulse wave. However, this is not the only possibility. A person skilled in the art can similarly construct a process flow even when other cases included in the present invention are adopted such as a case where the degree of fatigue is evaluated by using, as an index, change in waveform of at least one of wave components, a, b, c, d, and e in acceleration pulse wave, and a case where the degree of fatigue is evaluated by using, as an index, change of a ratio in measured value between at least two of wave components, a, b, c, d, and e in acceleration pulse wave, and the measured value is at least one of measured values: wave height, frequency, wavelength, cycle, and variation coefficient of the foregoing measured values.

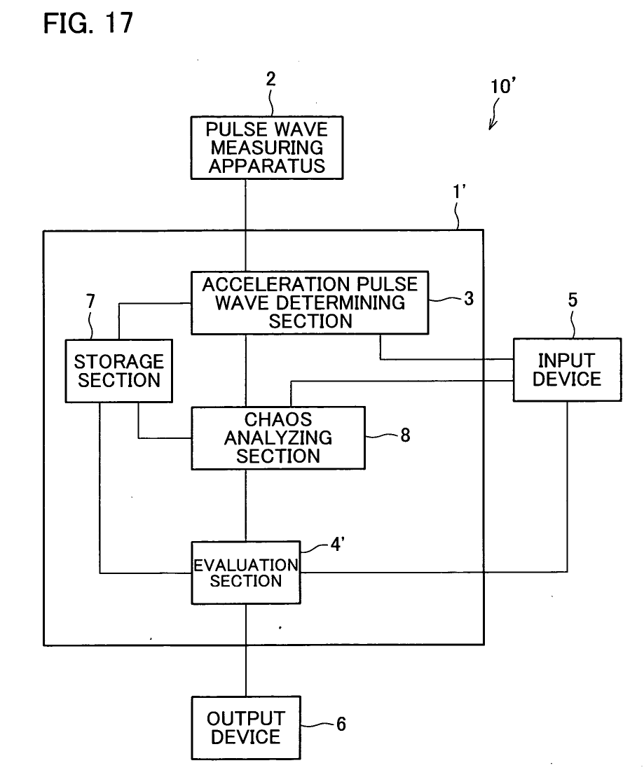

The following will describe another embodiment of a fatigue evaluation apparatus according to the present invention with reference to FIG. 17. Another fatigue evaluation system 10′ according to the present embodiment includes the pulse wave measuring apparatus 2, a fatigue evaluation apparatus 1′, the input device 5, and the output device 6. The fatigue evaluation apparatus 1′ includes the acceleration pulse wave determining section 3, an evaluation section 4′, the storage section 7, and a chaos analyzing section 8. Note that members except for the evaluation section 4′ and the chaos analyzing section 8 have the same functions as the foregoing members and devices, and the explanation thereof is omitted here. Only the evaluation section 4′ and the chaos analyzing section 8 which are features of the present embodiment are described here.

The chaos analyzing section 8 serves as chaos analyzing means for performing chaos analysis on acceleration pulse wave, and is means for performing the foregoing chaos analyses, i.e. “traditional chaos analysis using a combination of correlation dimension analysis and maximum Lyapunov exponent analysis” and/or “chaos analysis using maximum entropy method”.

Further, the evaluation section 4′ is evaluation means for evaluating the degree of fatigue by using, as an index, a factor in the chaos analysis obtained by the chaos analyzing section 8, and performs a method for evaluating the degree of fatigue from a result of the foregoing chaos analysis.

Specifically, the evaluation section 4′, if chaos analysis is “traditional chaos analysis using a combination of correlation dimension analysis and maximum Lyapunov exponent analysis”, evaluates the degree of fatigue based on maximum Lyapunov exponent or correlation dimension. For example, when maximum Lyapunov exponent is a factor in the chaos analysis as the index, the evaluation section 4′ compares a measured maximum Lyapunov exponent with a maximum Lyapunov exponent at the reference time, and if the measured maximum Lyapunov exponent is lower than the maximum Lyapunov exponent at the reference time, the evaluation section 4′ evaluates it as being indicative of fatigue. Further, when correlation dimension is a factor in the chaos analysis as the index, the evaluation section 4′ compares a measured correlation dimension with a correlation dimension at the reference time, if the measured correlation dimension is closer to an integral value than the correlation dimension at the reference time, the evaluation section 4′ evaluates it as being indicative of fatigue.

On the other hand, if chaos analysis is “chaos analysis using maximum entropy method”, the evaluation section 4′ evaluates the degree of fatigue by using a high-frequency component as a factor in the chaos analysis that is the index. In this case, the evaluation section 4′ compares a measured high-frequency component with a high-frequency component at the reference time, and if the slope of the measured high-frequency component is sharper than the slope of a high-frequency component at a reference time, the evaluation section 4′ evaluates it as being indicative of fatigue.

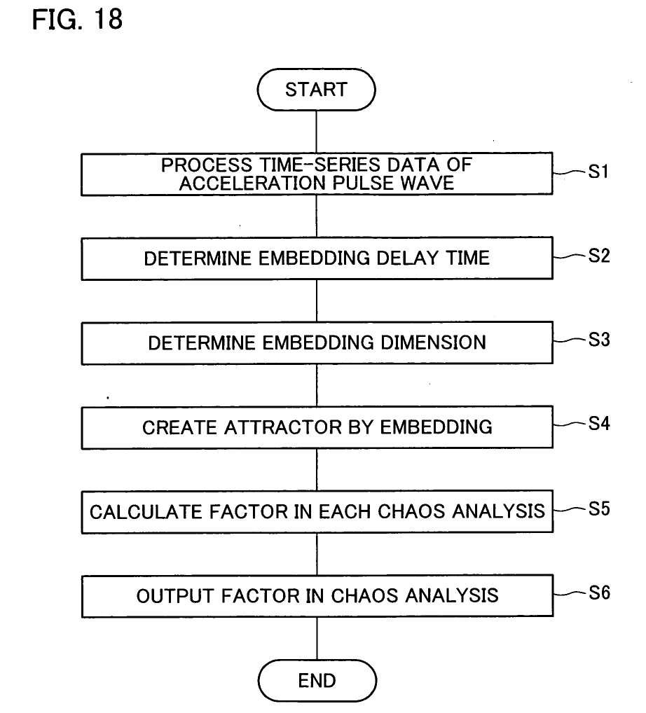

FIG. 18 illustrates one example of a specific process flow in the chaos analyzing section 8. In Step 1, the chaos analyzing section 8 processes time-series data of acceleration pulse wave obtained by the acceleration pulse wave determining section 3. In this step, it is determined whether a string of time-series from which noise portions and other portions have been removed has sufficient data length. In addition, the data is normalized if necessary. Further, a necessary portion may be extracted.

Next, in Step 2, the chaos analyzing section 8 determines an embedding delay time by using an autocorrelation function. Specifically, the autocorrelation function R(Δt) is calculated from the following equation (3):

$$R ( Δ t ) = \frac{1}{N} {\sum }_{Δ t = 1}^{N} x ( t ) x ( t + Δ t ) . \qquad ( 3 )$$

In a plot having x axis for Δt and y axis for R(Δt), a value of Δt when R(Δt) is initially 0, or a value of Δt when R(Δt) is the closest to a zero-crossing point is the embedding delay time.

Then, in Step 3, the chaos analyzing section 8 determines an embedding dimension. Examples of embedding dimension determining method include a correlation dimension method obtained from GP (Grassberger-Procaccia) algorithm, maximum-likelihood method, Judd's method, box-counting method. Any of the methods may be adopted. Note that, in the present embodiment, embedding dimension is determined by using the correlation dimension method as follows: In the above embedding delay time, temporary embedding is performed on various dimensions (from two dimensions to about twenty dimensions). As to a hypersphere having a radius r of a given point on an embedded attractor, a correlation dimension is determined by using a correlation integral (C(r)) calculated from GP (Grassberger-Procaccia) method, expressed by the equation (1).

From the thus obtained correlation dimension, an embedding dimension is determined.

Next, in Step 4, the chaos analyzing section 8 creates an attractor by embedding. Then, in Step 5, the chaos analyzing section 8 calculates a factor in each chaos analysis (chaotic parameter).

Finally, in Step 6, the chaos analyzing section 8 outputs a result of the calculation to the evaluation section 4′, and then finishes the process.

Here, the autocorrelation function in Step 2 will be described in detail. In the Equation (3), N is the number of samples. The autocorrelation function is found by calculation of a correlation function of time-series waveforms x(t) and x(t+Δt) with Δt increase. It is standardized that the autocorrelation function R(Δt) at a given Δt becomes 1 when x(t) and x(t+Δt) are in perfect agreement with each other, becomes −1 when they are in agreement with each other in applying sign inversion, and becomes 0 when they are not in agreement with each other.

The autocorrelation function represents the extent of loss similarity of variation with time. When x(t) has a periodicity (This case applies to time-series data of pulse wave, such as acceleration pulse wave, electrocardiogram, electroencephalogram, and the like), R(Δt) repeats increase and decrease with Δt. On the other hand, when there is white noise, R(Δt) becomes 0 even if Δt is few (even if a phase shifts only slightly).

The first zero crossing method of the autocorrelation function R(Δt) is adopted herein. Alternatively, there have been proposed a method of first using a minimal value of Δt for R(Δt), many methods using an autocorrelation function, and many methods not using an autocorrelation function. However, almost all chaos analysis methods currently adopt this autocorrelation function zero crossing method.

That is, in the technique shown in Examples described later (sampling rate of 10 msec), the embedding delay time is 4 to 6 steps. Of course, the value of the embedding delay time differs with change of a sampling rate (Embedding delay time differs substantially in proportional with the value of a sampling rate.). Therefore, it should be noted that the present invention includes cases when the setting conditions and processing conditions are changed as appropriate.

Further, determination of embedding dimension in Step 3 will be described in detail below.

<Correlation Dimension Method>

Determination of embedding dimension by the correlation dimension method is performed by calculation using the foregoing Equation (1). In this case, a distance between two points xi and xj is calculated. Here, the distance between two points may be defined as the Euclidean distance. Alternatively, arithmetic distance may be used most simply. It can be considered that other definition of the distance may be used, but unnecessarily increases a calculation time.

In a plot of a double logarithmic graph with the correlation integral (C(r)) calculated using the above equation, a scaling part is extracted from a graph of log r−log(C(r)) in each dimension. Then, its slope is calculated using a least squares method. The slope saturates with increase in number of dimensions. Therefore, more specifically, the embedding dimension is determined from a saturation value (correlation exponent). Note that, there are a plurality of ways to extract the scaling part and ways to determine a value at which the slope saturates, and the above ways are not the only possibilities.

<Embedding Dimension Determination Method Other than Correlation Dimension Method>

Currently, it is general to use the correlation dimension method as a method for determining an embedding dimension. However, other methods, i.e. maximum-likelihood method, Judd's method, box-counting method may be used. Thus, the embedding dimension determination method of the present invention is not limited to the correlation dimension method. It should be noted that, the box-counting method needs much calculation time, and thus is not favorable from a practical standpoint. In addition, it should be noted that the maximum-likelihood method has the problem of a slightly complex algorithm, but has an advantage that it is possible to accurately determine an embedding dimension even with a small amount of time-series data.

The above description has given an exemplary process flow of chaos analysis in the chaos analyzing section 8. However, the present invention is not limited to this. A person skilled in the art can similarly construct other process flow for performing chaos analysis.

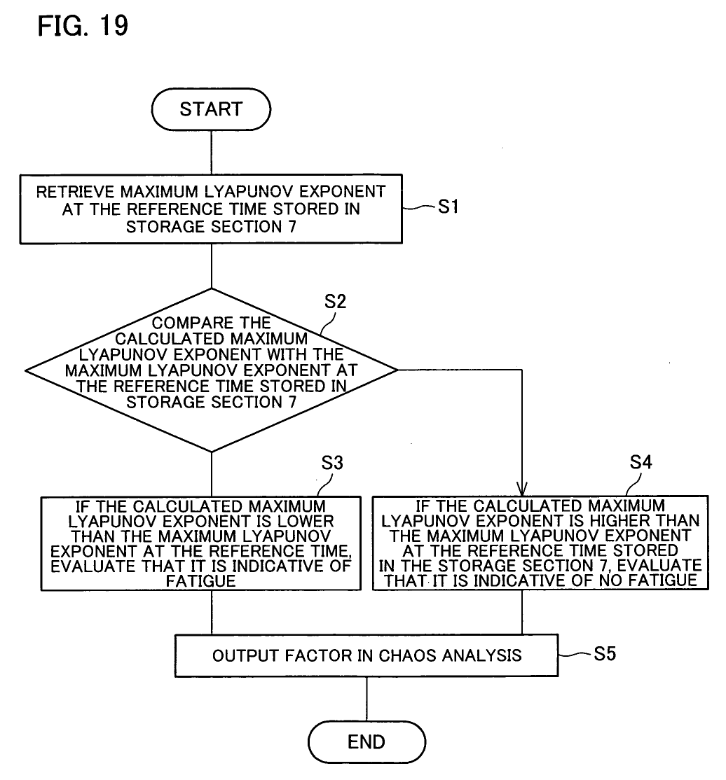

Next, FIG. 19 illustrates specifically an exemplary process flow in the evaluation section 4′. The following description will be given based on an exemplary process in a case where the maximum Lyapunov exponent is used as a factor in the chaos analysis.

First, in Step 1, the evaluation section 4′ retrieves the maximum Lyapunov exponent at the reference time stored in the storage section 7. Then, in Step 2, the evaluation section 4′ compares the calculated maximum Lyapunov exponent by the chaos analyzing section 8 with the maximum Lyapunov exponent at the reference time stored in the storage section 7.

The evaluation section 4′ evaluates, if the calculated maximum Lyapunov exponent is lower than the maximum Lyapunov exponent at the reference time stored in the storage section 7, that it is indicative of fatigue (Step 3). On the other hand, the evaluation section 4′ evaluates, if the calculated maximum Lyapunov exponent is higher than the maximum Lyapunov exponent at the reference time stored in the storage section 7, that it is indicative of no fatigue (Step 4).

Finally, the evaluation section 4′ outputs a result of the evaluation to the output device 6, and ends the process.

Note that, the above description has been given based on an exemplary process in a case where “traditional chaos analysis using a combination of correlation dimension analysis and maximum Lyapunov exponent analysis” is used as chaos analysis, and a maximum Lyapunov exponent is used as a factor in the chaos analysis. However, the present invention is not limited to this. For example, a person skilled in the art can similarly construct a process flow even when other cases are adopted such as a case where the correlation dimension is used as a factor in the chaos analysis, and a case where “chaos analysis using maximum entropy method” is used as chaos analysis and a high-frequency component is used as a factor in the chaos analysis that is an index.

As described above, by using a fatigue evaluation method and a fatigue evaluation apparatus according to the present invention, it is possible to evaluate the degree of fatigue simply, accurately, and objectively.

Note that, the present invention includes a fatigue evaluation method performing chaos analysis on acceleration plethysmogram to use a factor in the chaos analysis as an index. Calculation of various chaotic factors (chaotic parameters) has been described herein as an example based on a maximum Lyapunov exponent or correlation dimension. However, this is not the only possibility. Alternatively, methods such as Kolmogorov-Sinai (KS) entropy, recurrence plot, iso-directional recurrence plot, iso-directional neighbors plot, Higuchi's fractal dimension can be used. Among these methods, as a suitable method, preferable is to use a maximum Lyapunov exponent as a factor in the chaos analysis that is an index and evaluate a lower maximum Lyapunov exponent as being indicative of more fatigue. Further, preferable is to use a correlation dimension as a factor in the chaos analysis that is an index and evaluate a correlation dimension closer to an integral value as being indicative of more fatigue.

Still further, it is preferable to use the maximum entropy method in the chaos analysis. In this case, it is preferable to use a high-frequency component as a factor in the chaos analysis that is an index and evaluate the high-frequency component having a sharper slope as being indicative of more fatigue.

In Examples described later, the present invention is implemented by using a system of Artett (product name of plethysmograph) so as to measure a pulse wave. In the present Examples, data are collected from a fingertip for one minute at a sampling rate of 5 msec. However, the sampling rate may be 1 msec. In other words, conditions for the data collection at the time of pulse-wave measurement can be set as appropriate.

In addition, data collection for the pulse-wave measurement may be performed from any body parts where a pulse wave can be measured other than fingertip, such as earlobe, wrist, upper arm, or carotid. That is, as far as acceleration plethysmogram is obtained by double differentiation of the obtained pulse wave data, analysis of the degree of fatigue according to the present invention is possible.

It can be said that a high sampling rate (e.g. 1 mse) is preferable for data processing because the amount of information obtained increases at a high sampling rate. However, the higher the sampling rate, the greater the number of sets of data to be processed. This requires a huge number of calculations, and thus increases a calculation time of a personal computer (PC) with accelerating speed. In view of this, data obtained by thinning out by half data collected for one minute at a sampling rate of 5 msec is used herein. If a PC with a high computing speed can be prepared, data thinning is not necessary. This is because thinning of data decreases the amount of information in this data.

Note that, components and process steps of a fatigue evaluation apparatus of the foregoing embodiment can be realized by a CPU or other computing means executing a program contained in a ROM (Read Only Memory), RAM, or other storage medium for controlling input means such as a keyboard, output means such as a display, and communications means such as an interface circuit. Therefore, it is possible to realize various functions and various processes of a fatigue evaluation apparatus of the present embodiment only by a computer having these means, reading a storage medium storing the program and executing the program. In addition, it is possible to realize various functions and various processes on any computer with the use of a removable storage medium storing the program.

The storage medium may be a memory (not shown) for process steps on a microcomputer. For example, the program medium may be something like a ROM. Alternatively, the program medium may be such that a program reader device (not shown) as an external storage device may be provided in which a storage medium is inserted for reading.

In addition, in any case, the stored program is preferably executable on access by a microprocessor. Further, it is preferred if the program is retrieved, and the retrieved program is downloaded to a program storage area in a microcomputer to execute the program. The download program is stored in a main body device in advance.

In addition, the program medium may be a storage medium constructed separably from a main body. The medium may be tape based, such as a magnetic tape or cassette tape; disc based, such as a flexible disc or hard disk including a magnetic disc and CD/MO/MD/DVD; card based, such as an IC card (including a memory card); or a semiconductor memory, such as a mask ROM, EPROM (Erasable Programmable Read Only Memory), EEPROM (Electrically Erasable Programmable Read Only Memory), and a flash ROM. All these types of media hold the program in a fixed manner.

In contrast, if the system is arranged to connect to the Internet or another communication network, the medium is preferably a storage medium which holds the program in a flowing manner so that the program can be downloaded over the communication network.

Further, if the program is downloaded over a communication network in this manner, it is preferred if the download program is either stored in a main body device in advance or installed from another storage medium.

Further, as to the chaos analyzing section, a separate chaos analyzing chip may be externally connected (inserted) to a PC, so as to constitute a fatigue evaluation apparatus. That is, the chaos analyzing section may be a chaos analyzing chip that is a device (equipment) specialized in chaos analysis process according to the present invention, which is different from a general-purpose CPU installed in a typical PC. With the chaos analyzing chip, it can be expected to realize a super-computer-level computing speed even in a typical PC. In addition, after pulse-wave measurement, chaos analysis can be performed promptly and reliably on a result of the measurement. Therefore, the present invention includes a chaos analyzing device that is a device derived from the chaos analyzing section (chaos analyzing means) for performing the foregoing chaos analysis.

<Method for Collecting Data which is an Object to be Evaluated for the Degree of Fatigue>

Viewing from another aspect, the present invention relates to a method for collecting data which is an object to be evaluated for the degree of fatigue, wherein change of a measured value of at least one of wave components a, b, c, d, and e in acceleration pulse wave is measured, and the measured value may be at least one of measured values: wave height, frequency, wavelength, cycle, and variation coefficient of the foregoing measured values. Preferably, the present invention relates to a method for collecting data which is an object to be evaluated for the degree of fatigue, wherein change in a wave height of at least one of wave components a, b, c, d, and e in acceleration pulse wave is measured. According to the method, change in a wave height is treated as data which is an object to be evaluated for the degree of fatigue.

<Database>

As another mode, the present invention relates to a database which includes a measured value of at least one of wave components a, b, c, d, and e in acceleration pulse wave at a reference time, and the measured value is at least one of measured values: wave height, frequency, wavelength, cycle, and variation coefficient of the foregoing measured values. Preferably, the present invention relates to a database which includes a wave height of at least one of the wave components a, b, c, d, and e in acceleration pulse wave at a reference time. Necessary information at a reference time when a certain subject has no fatigue is stored in a database, which brings together data obtained in this manner in the form of digitized information. For comparison with data obtained from a subject by an evaluation method of the present invention, a database containing data in the form of digitized information provided in a computer or the like can be used easily and simply. In this mode, the present invention offers an apparatus which is provided with the database of the present invention.

<Fatigue Evaluation Method Using a Database>

As still another mode, the present invention relates to a fatigue evaluation method for evaluating the degree of fatigue by using, as an index, change in waveform of at least one of wave components a, b, c, d, and e in acceleration pulse wave, wherein it is evaluated that a wave height lower than wave height data stored in a database of the present invention is indicative of fatigue.

The present invention is the result of “Research into the molecular and nervous system mechanisms for fatigue and feelings of fatigue, and into their prevention” by special coordination funds for promoting science and technology in Ministry of Education, Culture, Sports, Science and Technology of Japan.

### EXAMPLES

The following will describe details of the present invention in accordance with Examples. However, the present invention is not limited by the Examples.

### Example 1

<Fatigue Evaluation>

Effects of Suppressing Decrease in Working Efficiency

(1) Test Subjects

Mental workloads of improved ATMT were given to six normal males aged 20-29 for evaluation of a wave height of the wave component a in human acceleration pulse wave before and after mental work loading. The acceleration pulse wave was determined by using an acceleration plethysmogram determination system called Artett (U-Medica Inc.). This test was conducted on the same subject for mutually close two days in a week by a blind method. In this test, habitual users of caffeine-containing foods (e.g. coffee, health drink, and gum) and drugs having effects on the central nervous system, such as antiallergic agent and their users on the test date were excluded.

(2) Ways of Mental Work Loading

ATMT (Advanced Trail Making Test) is a test used for evaluation of aging phenomena and screening of early dementia. The ATMT gives the test subjects a visual search task for quickly touching numbers 1 to 25 presented on a touch panel display. Unlike conventionally conducted TMT (test of giving a task of sequentially drawing lines on numbers 1 to 25 randomly distributed on a sheet of A4 paper in a manner similar to one stroke drawing), the ATMT can measure a search response time for each target number, rearrange all the target numbers for each response, and create a new target number with the pointed targets made disappeared from the display. With this arrangement, it is possible to evaluate an increased mental fatigue shown during the task and application of working memory for enhancing search efficiency, for example. In the ATMT, numbers appeared on a screen of the display are arranged in three patterns, A, B, and C. In the pattern A, when a target button is touched, the target button changes in color, which differentiates the touched target button from other buttons. In pattern B, when a target button is touched, the touched target button disappears and other number appears so that 25 numbers are arranged on the screen. In pattern C, when a target button is touched, a number on the touched target button disappears, and other number disappears on the next screen with 25 numbers arranged randomly for each time. After completion of the task of touching all the numbers in these three patterns, a computer calculates a time taken for the task. These three patters makes up one set of the task (Japanese Laid-Open Patent Application No. 112981/2002; Tokukai 2002-112981; published on Apr. 16, 2002).

Here, as to mental work loading, 50 numbers from 20 to 69 were used as numbers of target buttons, and mental work loading was performed with the improved ATMT which is similar to the foregoing ATMT, except for calculation of the time taking for the task. That is, mental workload was given in such a manner that tests A, B, and C were continuously repeated for four hours in the morning. To motivate the subjects, it was informed to the subjects that the subjects can get a reward every time they complete each pattern.

(3) Result

As a result of measurement of a wave height of the wave component a in acceleration pulse wave, the wave component a's wave height of 361.3 before the start of the improved ATMT, significantly decreased to 129.9 after completion of the ATMT (P<0.005) (FIG. 2). FIG. 2 indicates that a wave height of the wave component a significantly changes after and before fatigue loading, and that a wave height of the wave component a decreases from fatigue.

### Example 2

<Fatigue Evaluations Under Mental Fatigue Loads and Physical Fatigue Loads>

(1) Test Subjects for a Fatigue Load Test

Mental fatigue loads and physical fatigue loads were given to five normal males aged 20-29 (aged 24.8±2.0), acceleration pulse waves before and after fatigue loading were determined, and chaos analyses were performed on the determined acceleration pulse waves. In contrast with the test subjects under fatigue loads, a control group spent in a relaxed state, without performing tasks.

The present fatigue load test was conducted in compliance with an ethical principle based on the Helsinki Declaration. The present fatigue load test was conducted after the test was examined in advance on the execution plan of the test, qualification of a doctor responsible for the test, and others by “the Committee of Judgment as to Commission of Clinical Tests for Specified Health Foods in Soiken Inc. and Soiken Clinic” (hereinafter referred to as “clinical test judging committee”), and was approved for conduct of the test by the clinical test judging committee. Voluntary written consents to participation in the present test were obtained from the subjects before start of the test.

(2) Ways of Fatigue Loading

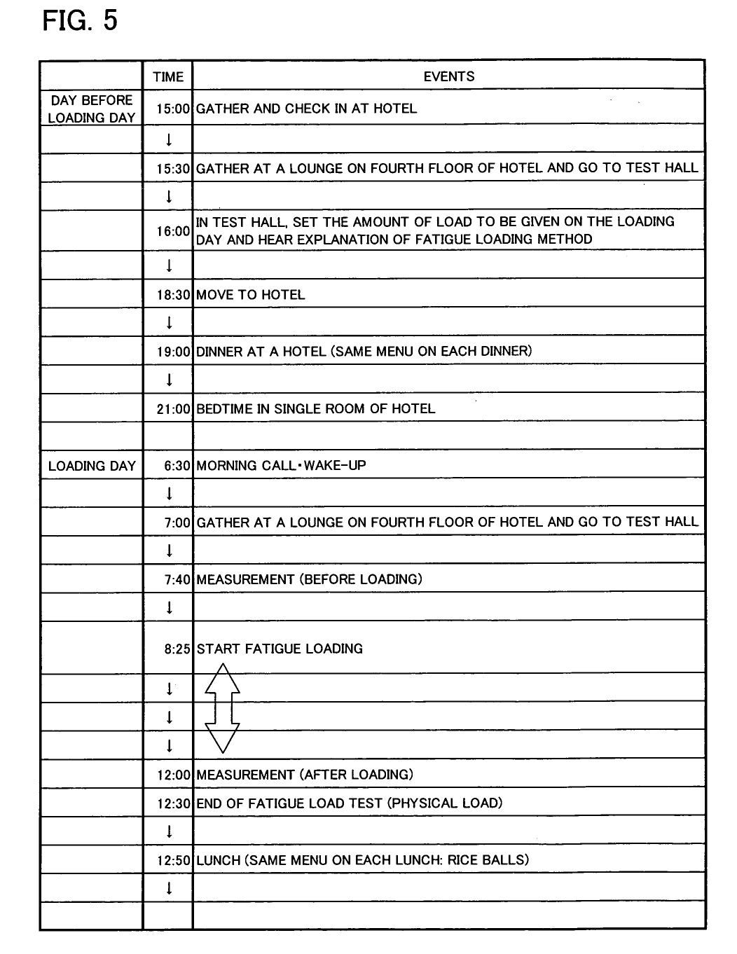

For the purpose of comparisons between a state under mental fatigue and a controlled state and between a state under physical fatigue and a controlled state, a three-period crossover test (in open study) was conducted on each of the subjects. A brief outline of the fatigue load test is illustrated in FIG. 5. To minimize differences in environmental condition between the subjects during the test period, the subjects were instructed to keep in mind the following things.

(Before Test Period)
No binge eating and drinking and no excessive exercises for three days in advance of the test
Keep a record of diets for three days before the test
Check on the degree of fatigue in a daily life before the first test of each schedule with questionnaire on lifestyle and questionnaire on subjective symptoms including VAS (note 1), Face Scale (note 2), and fatigue scale (note 3)

(During Test Period)
Do activities of daily life, such as having meals, taking a bath, and sleeping, according to the test schedule
Take meals of the following menus:

Dinner Menus on the day before the task, loading: Chicken steak with Japanese-style source

Lunch Menu on the day of the task loading: three rice balls
Only a mineral water is permitted for drink. The intake of water is not limited.

(Care During a Period Between Tests)
No vitamin supplements taking, smoking, blood donation, and other activities applied to an exclusion criteria

(Note 1) VAS

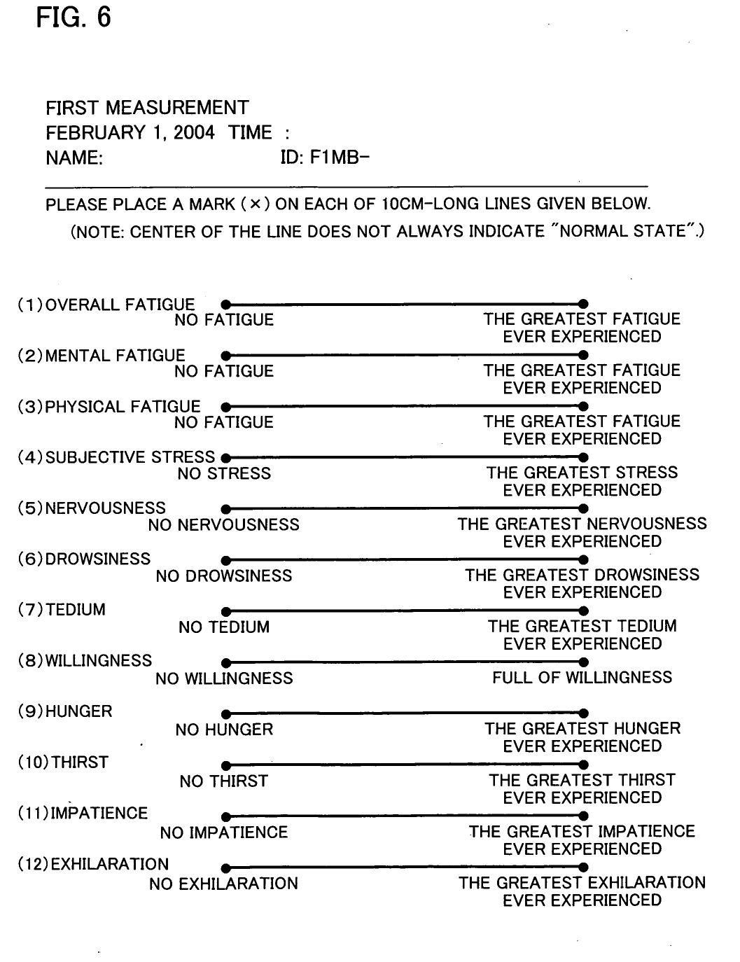

VAS is generally conducted to know a subjective fatigue of a subject, and is an evaluation method in which each subject is shown a line segment written on a sheet with expressions of criteria for a target variable at both ends of the line segment, and then asked to mark on the line segment where the target variable lies. An advantage of the method is that a quantitative answer to a question about the target variable can be obtained by measuring how far the target variable is from the left end of the line segment, so that answers obtained from many people can be averaged out. FIG. 6 illustrates a VAS test sheet.

(Note 2) Face Scale

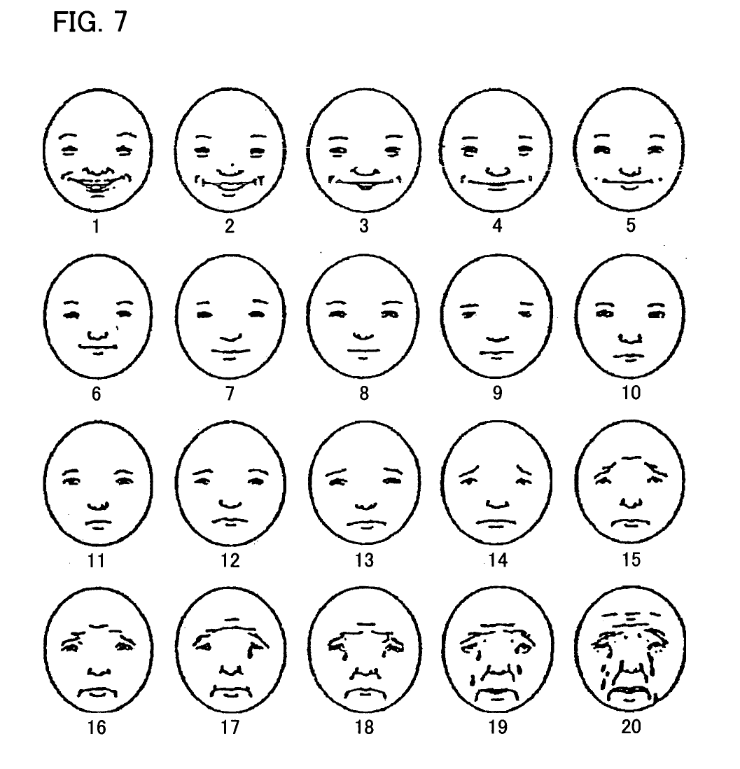

Face Scale is used as a scale for knowing a feeling of a subject on the day of the test, is a test in which a subject selects one picture representing a feeling of the subject from among face pictures of twenty levels from Level 1 (smiling face) to Level 20 (sad face). FIG. 7 illustrates a Face Scale test sheet.

(Note 3) Fatigue Scale

Fatigue scale is a test using a questionnaire on the degree of fatigue of a subject, and the subject answers the questionnaire.

(2)-(i) Ways of Mental Fatigue Loading

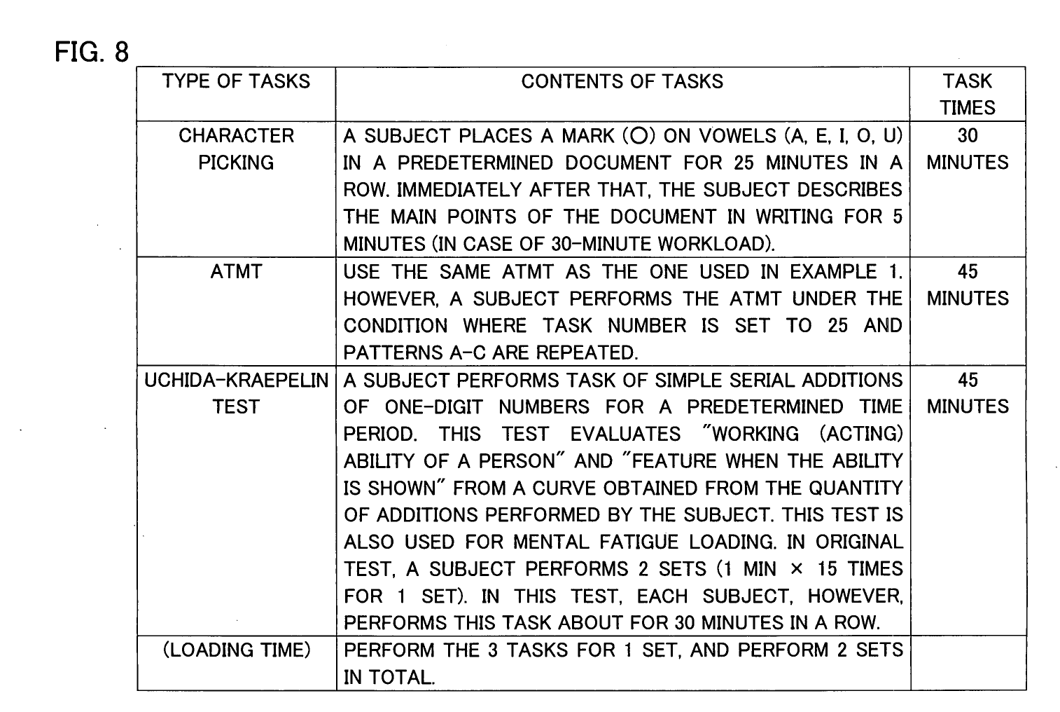

According to a set of fatigue loads illustrated in FIG. 8, subject was subjected to mental fatigue loading. In the present test, the subject was subjected to two sets of fatigue loads in FIG. 8.

(2)-(ii) Ways of Physical Fatigue Loading

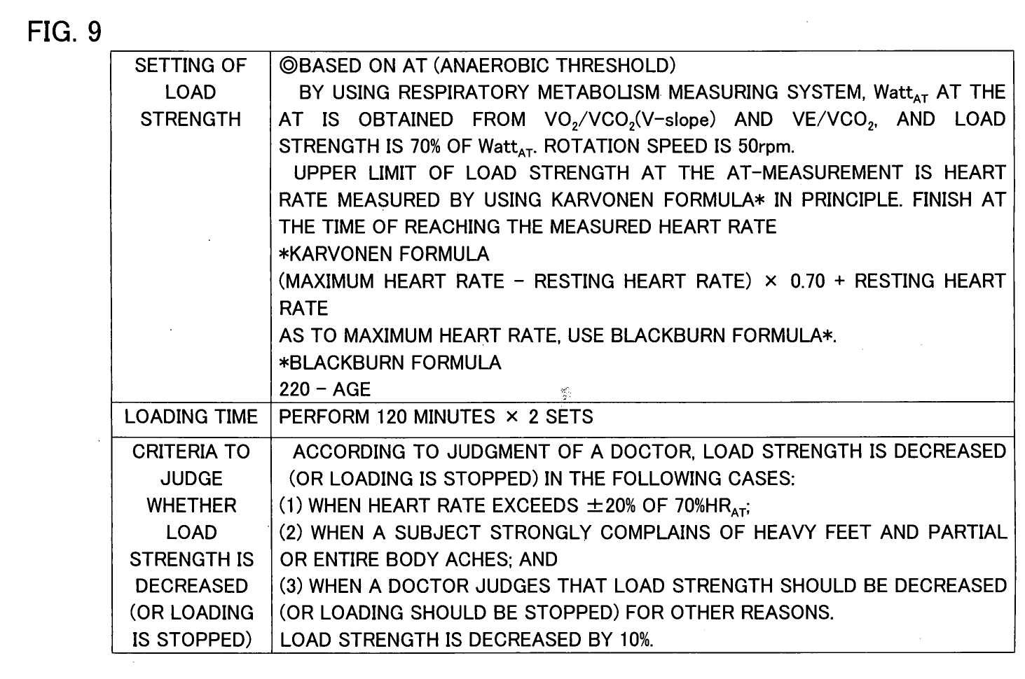

As illustrated in FIG. 9, a physical load strength was set for each subject on the day before the fatigue load test. An adopted equipment was respiratory metabolism measuring system (respiratory metabolism measuring apparatus: Aeromonitor AE-300S produced by Minato Medical Science Co., Ltd.; and fatigue loading apparatus: Aerobike 75XL ME produced by Combi Corporation). On the day of the fatigue load test, the subject was subjected to two sets of physical fatigue loads by using an ergometer.

(2)-(iii) No Loading

To minimize all loads given to the subjects, prepared for the subjects were separate rooms for men and women. Each of the rooms had an air bed, cushions, magazines, a DVD showing system, and others. The subjects are left to do what they like. However, the subjects are prohibited from sleeping.

(3) Measurements Before and After Fatigue Loading

(3)-(a) Evaluation of a Subjective Fatigue

To evaluate a subjective fatigue of the subject before and after the subject suffers from fatigue, the survey was conducted by the questionnaire on subjective symptoms including VAS, Face Scale, and fatigue scale.

(3)-(b) Measurement of Acceleration Pulse Wave

To use acceleration pulse wave adopted in the foregoing Example 1, acceleration pulse wave was measured before and after the fatigue loading. The measurement time was 60 seconds.

(4) Chaos Analysis of Acceleration Pulse Wave

Chaos analysis was performed in the following procedure:

(i) Adopt 4096 steps (10 msec for one step) from acceleration pulse wave time-series data (200 Hz);

(ii) Determine an embedding delay time using autocorrelation function, ranging from 4 to 6 steps;

(iii) Determine an embedding dimension using a correlation dimension method obtained from the GP algorithm (The embedding dimension is 4, but 3 or 5 is adopted for some data);

(iv) create an attractor by embedding; and

(v) Calculate each chaotic parameter (factor).

Test Results

(5)-(a) Result of Evaluation of Subjective Fatigue

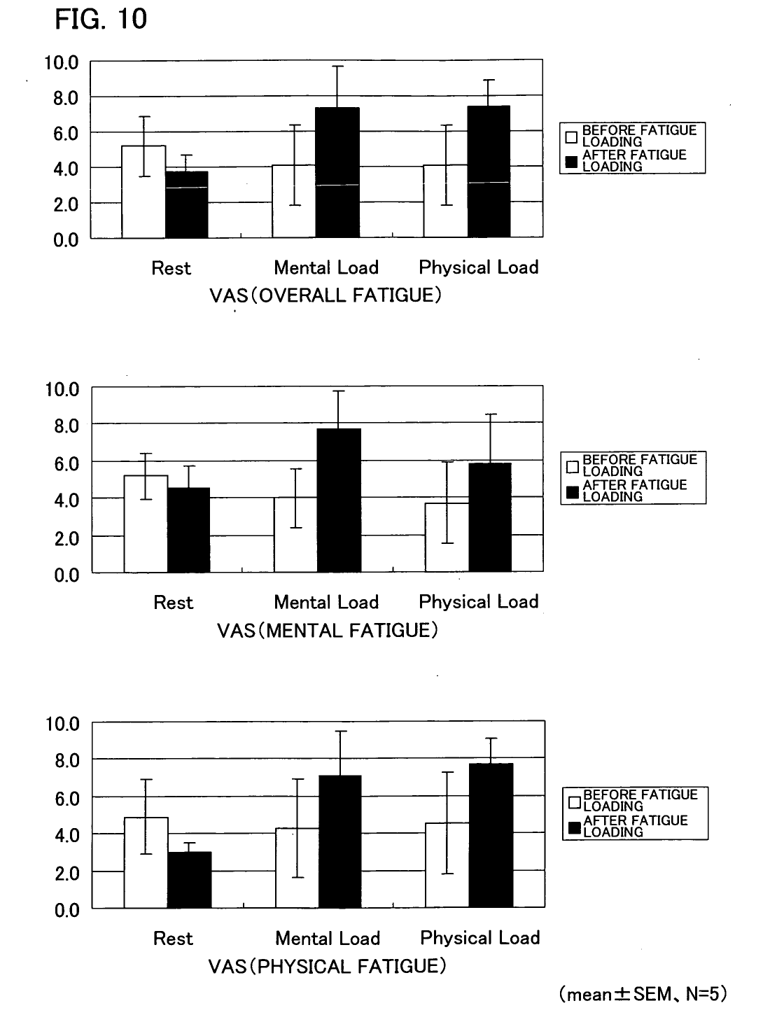

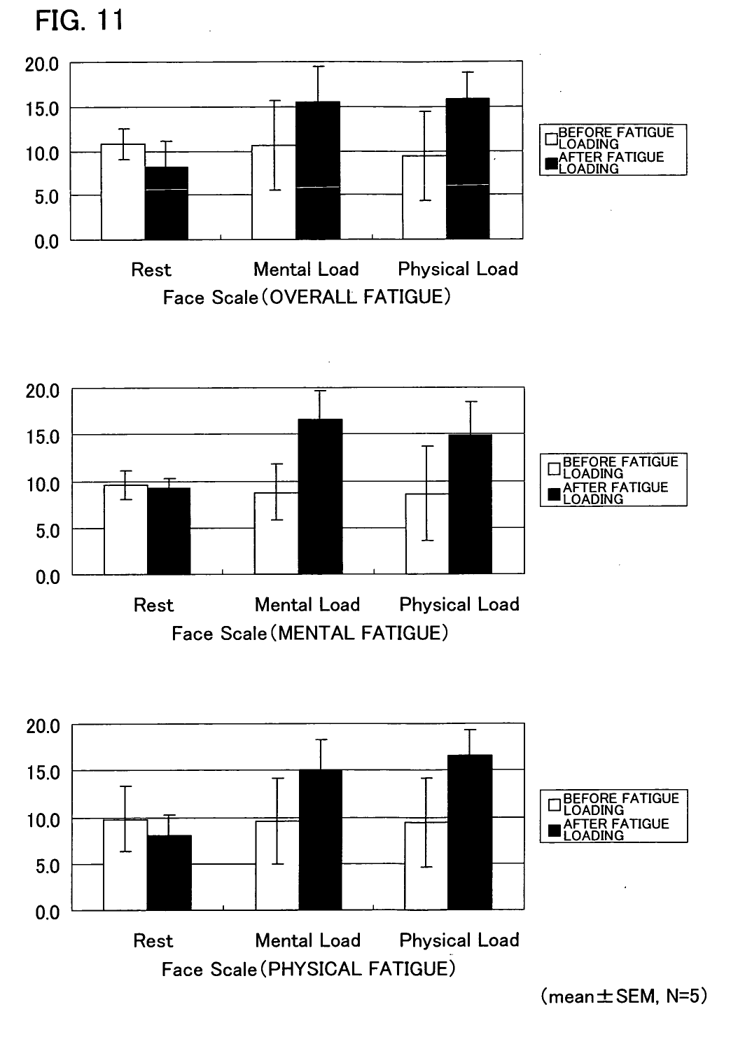

Results of evaluation of a subjective fatigue before and after fatigue loading are shown in FIG. 10 (VAS) and FIG. 11 (Face Scale). A subjective fatigue was observed after mental and physical fatigue loading.

(5)-(b) Result of Chaos Analysis of Acceleration Pulse Wave

(5)-(b-i) Lyapunov Exponent

FIG. 12 illustrates changes in maximum Lyapunov exponent before and after fatigue loading. As a result of comparison in maximum Lyapunov exponent between the conditions before and after mental fatigue loading, the maximum Lyapunov exponent increased significantly after mental fatigue loading (P<0.02).

(5)-(b-ii) Correlation Dimension

FIG. 13 illustrates changes in correlation dimension before and after fatigue loading. Significant difference was not observed in correlation dimension between before and after mental and physical fatigue loading. However, a tendency of a non-integral value turning to an integral value was observed. This means that a similar waveform is repeated, and fatigue loads make the waveform more monotone. Considering this with the foregoing result of the maximum Lyapunov exponent, it can be considered that chaotic nature reduces due to fatigue loads.

(5)-(b-iii) Slope of High-Frequency Component in Chaos Analysis Using Maximum Entropy Method

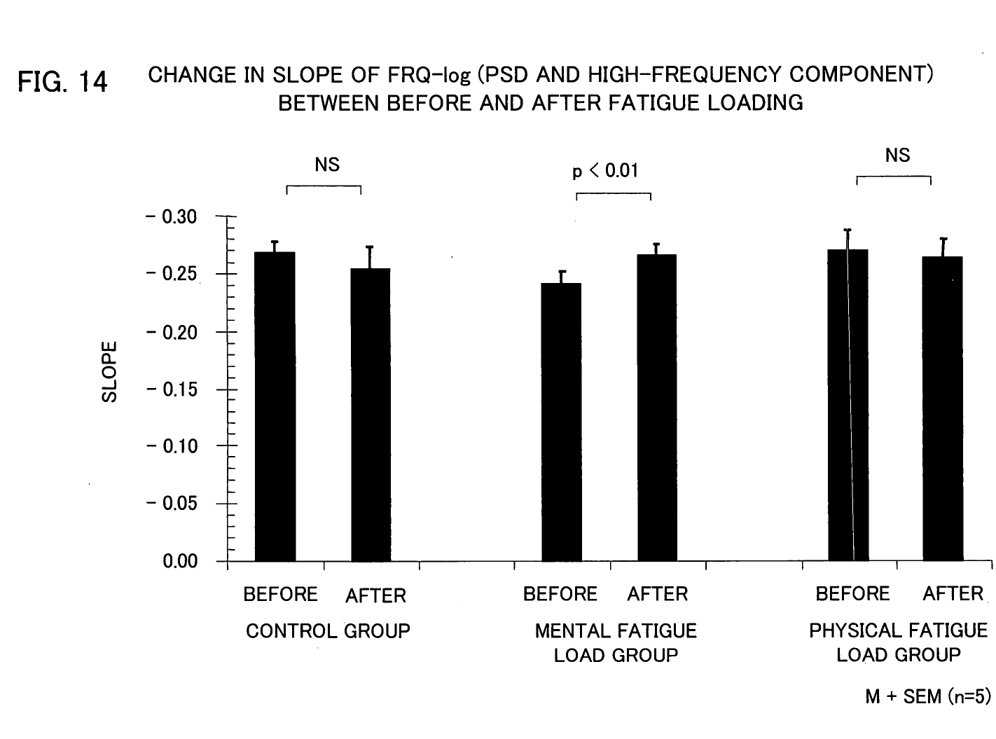

FIG. 14 illustrates change in slope of a high-frequency component before and after fatigue loading. As a result of comparison in slope of a high-frequency component between before and after mental fatigue loading, it was indicated that the slope significantly increased after mental fatigue loading (P<0.01).

Specific embodiments or examples implemented in the description of the BEST MODE FOR CARRYING OUT THE INVENTION only show technical features of the present invention and are not intended to limit the scope of the invention. Variations can be effected within the spirit of the present invention and the scope of the following claims.

### INDUSTRIAL APPLICABILITY

According to the above arrangement, it is possible to measure the degree of fatigue easily and quantitatively. Quantification of the degree of fatigue is important in terms of prevention of chronic fatigue syndrome and overwork death.

In addition, quantification of the degree of fatigue can serve as a method for early detecting fatigue to prevent the occurrence of serious accidents caused by mistake due to fatigue (e.g. aircraft accident, accident on high-speed mass transportation, accident on nuclear power plant, and various medical accidents), or a method for early detecting fatigue to offer a respite before manifestation of a decreased working efficiency, and is important as a method for preventing decrease of productivity in industrial worksites.

1. A fatigue evaluation method, wherein change in waveform of a pulse wave is an index.

2. The method according to claim 1, wherein the pulse wave as an index is an acceleration pulse wave.

3. The method according to claim 2, wherein a waveform of the acceleration pulse wave as the index is a waveform of at least one of wave components a, b, c, d, and e.

4. The method according to claim 3, wherein change in waveform of the acceleration pulse wave as an index is change of a measured value of at least one of the wave components a, b, c, d, and e in the acceleration pulse wave, and the measured value of the wave component is at least one of measured values: wave height, frequency, wavelength, cycle, and variation coefficient of the foregoing measured values.

5. The method according to claim 4, wherein the change in waveform of the acceleration pulse wave is change in wave height of the wave component a.

6. A fatigue evaluation method using, as an index, change in waveform of at least one of wave components, a, b, c, d, and e in acceleration pulse wave, wherein
it is evaluated that a wave height lower than a wave height at a reference time is indicative of fatigue.

7. The method according to claim 6, wherein
the index is change in wave height of the wave component a.

8. The method according to claim 2, wherein change in waveform of the acceleration pulse wave as an index is change of a ratio in measured value between at least two of the wave components a, b, c, d, and e in the acceleration pulse wave, and the measured value of the wave component is at least one of measured values: wave height, frequency, wavelength, cycle, and variation coefficient of the foregoing measured values.

9. The method according to claim 2, wherein chaos analysis is performed on the acceleration pulse wave so that a degree of fatigue is evaluated by using, as an index, change of a factor in the chaos analysis.

10. The method according to claim 9, wherein the factor in the chaos analysis used as the index is a maximum Lyapunov exponent, and it is evaluated that the maximum Lyapunov exponent lower than a maximum Lyapunov exponent at a reference time is indicative of fatigue.

11. The method according to claim 9, wherein the factor in the chaos analysis used as the index is a correlation dimension, and it is evaluated that the correlation dimension closer to an integral value than a correlation dimension at a reference time, is indicative of fatigue.

12. The method according to claim 9, wherein a maximum entropy method is used in the chaos analysis.

13. The method according to claim 12, wherein the factor in the chaos analysis used as the index is a high-frequency component, and it is evaluated that the high-frequency component having a sharper slope than a high-frequency component at a reference time is indicative of fatigue.

14. The method according to claim 1, wherein a pulse wave obtained from a subject is used.

15. (canceled)

16. A fatigue evaluation apparatus comprising:
evaluation means for evaluating a degree of fatigue by using, as an index, change in waveform of acceleration pulse wave determined on the basis of a pulse wave obtained from a subject.

17. The apparatus according to claim 16, further comprising acceleration pulse wave determining means for determining acceleration pulse wave by twice differentiating the pulse wave obtained from the subject.

18. The apparatus according to claim 16, wherein the evaluation means evaluates the degree of fatigue by using, as an index, change in waveform of at least one of wave components a, b, c, d, and e.

19. The apparatus according to claim 18, wherein the evaluation means evaluates the degree of fatigue by using, as the index, change of a measured value of at least one of wave components a, b, c, d, and e in the acceleration pulse wave, and the measured value of the wave component is at least one of measured values: wave height, frequency, wavelength, cycle, and variation coefficient of the foregoing measured values.

20. The apparatus according to claim 16, wherein the evaluation means evaluates the degree of fatigue by using, as the index, change of a ratio in measured value between at least two of wave components a, b, c, d, and e in the acceleration pulse wave, and the measured value of the wave component is at least one of measured values: wave height, frequency, wavelength, cycle, and variation coefficient of the foregoing measured values.

21. A fatigue evaluation apparatus comprising:
chaos analyzing means for performing chaos analysis on an acceleration pulse wave determined on the basis of a pulse wave obtained from a subject; and
evaluation means for evaluating the degree of fatigue by using, as an index, change of a factor in the chaos analysis.

22. The apparatus according to claim 21, wherein the factor in the chaos analysis used as the index is a maximum Lyapunov exponent, and the evaluation means evaluates that the maximum Lyapunov exponent lower than a maximum Lyapunov exponent at a reference time is indicative of fatigue.

23. The apparatus according to claim 21, wherein the factor in the chaos analysis used as the index is a correlation dimension, and the evaluation means evaluates that the correlation dimension closer to an integral value than a correlation dimension at a reference time, is indicative of fatigue.

24. The apparatus according to claim 21, wherein the analyzing means uses a maximum entropy method in the chaos analysis.

25. The apparatus according to claim 24, wherein the factor in the chaos analysis used as the index is a high-frequency component, and the evaluation means evaluates that the high-frequency F component having a sharper slope than a high-frequency component at a reference time is indicative of fatigue.

26. A method for collecting data which is an object to be evaluated for the degree of fatigue, wherein change of a measured value of at least one of wave components a, b, c, d, and e in acceleration pulse wave is measured, and the measured value is at least one of measured values: wave height, frequency, wavelength, cycle, and variation coefficient of the foregoing measured values.

27. (canceled)

28. (canceled)

29. (canceled)

30. (canceled)
